(19) United States
(12) Patent Application Publication
(10) Pub. No.: US 2011/0275717 A1
(43) Pub. Date: Nov. 10, 2011

(54) PHARMACEUTICAL FORMULATION COMPRISING DICLOFENAC

(75) Inventor: Michele Virno, Roma (IT)

(73) Assignee: ADVANCE HOLDINGS LIMITED, Floriana (MT)

(21) Appl. No.: 13/128,747

(22) PCT Filed: Nov. 11, 2009
(86) PCT No.: PCT/EP2009/064971

§ 371 Date: Jul. 19, 2011

(30) Foreign Application Priority Data
Nov. 28, 2008 (EP) 08170177.3

(51) Int. Cl. A61K 31/196; A61P 19/02; A61P 29/00

(52) U.S. Cl. 514/567

(57) ABSTRACT

The present invention relates to a pharmaceutical formulation comprising a pharmaceutically acceptable salt of diclofenac, at least one polyoxyalkylene ester of a hydroxy fatty acid, water, and, optionally, a co-solvent.

### FIELD OF THE INVENTION

The present invention relates to a pharmaceutical formulation comprising diclofenac.

More in particular, the present invention relates to a pharmaceutical formulation comprising an aqueous solution of diclofenac for topical application having improved permeation and bioavailability properties.

### BACKGROUND OF THE INVENTION

Diclofenac is a non-steroidal anti-inflammatory drug (“NSAID”) known chemically as 2-[(2,6-Dichlorophenyl)amino]phenylacetic acid. Diclofenac belongs to the acetic acid class of NSAID. The drug was developed in the 1960s by scientists at Ciba-Geigy and is sold around the world by Novartis under various trade names, including Cataflam™ and Voltaren™ in Europe and United States.

Owing to its excellent analgesic properties, diclofenac is widely used for treating various types of pain, including both chronic and acute painful episodes. The drug is administered for the treatment of musculoskeletal and joint disorders such as rheumatoid arthritis, osteoarthritis, and ankylosing spondylitis; periarticular disorders such as bursitis and tendonitis; soft tissue disorders such as sprains and strains, and other painful conditions such as those following some surgical procedures.

Diclofenac is generally taken orally in the form of normal tablets or tablets covered with coatings resistant to gastric juices, or rectally, or by injection, or topically. Oral administration of diclofenac can cause serious adverse effects such as gastrointestinal bleeding and ulceration, liver and kidney damages, and central nervous system and cutaneous disturbances, particularly after extended use.

Therefore, in an effort to minimize the adverse effects associated with oral administration, non-oral delivery of diclofenac has been extensively investigated in recent years.

Topical formulations are attractive options because they avoid the hepatic first-pass metabolism, reduce the side effects associated with oral administration, are associated with higher patient compliance and, in some cases, enhance therapeutic efficacy of the drug.

However, the effectiveness of topical administration of diclofenac is limited by the difficulty of this drug to permeate the skin and by the low solubility of diclofenac in water.

Several patents and patent applications attempted to solve the above mentioned problems by means of gel topical formulations containing several ingredients to improve the solubility of diclofenac.

U.S. Pat. No. 4,711,906 discloses a liquid diclofenac preparation, in particular, for the parenteral application, consisting of a solution of diclofenac or one of its salts and, if desired, further pharmaceutical active ingredients and auxiliary substances in a solvent, the solvent consisting of 10-70 weight % preferably 20-50 weight %, of a mixture of (a) propylene glycol and (b) polyethylene glycol and 90-30 weight %, preferable 80-50 weight % of water, and in the solvent mixture the weight ratio of proylene glycol:polyethylene glycol being between 9.5:0.5 and 0.5:9.5, preferably between 3:1 and 1:3, especially preferably between 2:1 and 1:2.

U.S. Pat. No. 4,917,886 discloses a topically administrable pharmaceutical composition containing, as active ingredient, from approximately 0.1 to approximately 10% by weight of a non-steroidal, anti-inflammatorily active compound having at least one acidic group, from approximately 10 to approximately 50% by weight of a water-soluble, volatile lower alkanol having from 2 up to and including 4 carbon atoms, from approximately 3 to approximately 15% by weight of an optionally self-emulsifying lipid or a mixture of lipids, from approximately 0.5 to approximately 2% by weight of a gel structure former, from approximately 1 to approximately 20% by weight of a co-solvent, from approximately 40 to approximately 80% by weight of water, optionally from approximately 0.5 to approximately 5% by weight of an emulsifier if the lipid phase is not self-emulsifying and, if desired, non-essential constituents.

EP 147,476 discloses a gel preparation for external application characterized by being prepared from diclofenac sodium as the active ingredient, water, lower alkanols and glycols as medium, a carboxyvinyl polymer as gelating agent and a weak basic substance as neutralizing agent. The gel preparations for external application of this invention have good stability and nice feeling on use and show excellent anti inflammatory and analgesic effects by cutaneous absorption.

EP 488,089 discloses a diclofenac preparation for topical application which is packed together with a propellant gas in a compressed gas container and can be foamed from this through an atomiser with the aid of the propellant gas and delivered as diclofenac-containing foam.

EP 600,395 discloses an antiinflammatory and analgesic gel preparation comprising diclofenac or its salts, an ester of dibasic acid, a lower alcohol, and a nonionic polymer or a mixture of nonionic polymers selected from the group consisting of (a) 1.5-4% by weight of hydroxypropyl cellulose having a molecular weight of 500,000 or greater, (b) 2-4% by weight of hydroxyethyl cellulose having a molecular weight of 1,250,000 or greater, and (c) 1.5-4% by weight of a mixture of hydroxypropyl cellulose having a molecular weight of 500,000 or greater and hydroxyethyl cellulose having a molecular weight of 1,250,000 or greater, and having a viscosity of 5,000-35,000 cps and an yield value of 5 dyn/cm² or greater.

EP 788,794 discloses an external preparation composition, such as a liquid preparation, cream, ointment or cataplasm plaster, characterized in that the composition contains a water-soluble salt of diclofenac such as diclofenac sodium, water, and a fatty acid dialkylolamide and/or its polyoxyethylene adduct.

EP 834,312 discloses a medicament based on diclofenac or its salts, for topical treatment of inflammation and pain, containing at least one solvent and at least one solubiliser. The solvent is a mixture of water, diethyleneglycol monoethyl ether and optionally C2-6 polyalcohols and polyesters thereof, the esters and ethers thereof, as well as glycerides and/or ethoxylated derivatives thereof. The solubiliser is at least one phospholipid.

EP 1,003 499 discloses a pharmaceutical preparation for topical application containing diclofenac as an active substance which is dissolved in a solvent mixture, containing at least one alkyl alcohol with 2 to 4 C atoms as a main constituent, at least one short-chain N alkyl pyrrolidone and at least one pyrrolidone substituted with a long-chain alkyl radical.

US2005/239894 discloses a pharmaceutical composition intended for topical use comprising (a) 0.02-0.4% (w/w) of diclofenac sodium salt, (b) at least 50% (w/w) of water, (c) 0-30% (w/w) of at least one C2-C4-alkanol, (d) 3-20% (w/w) of a glycol solvent selected from the group consisting of propylene glycol and polyethylene glycol (200-20000), (e) 0.2-3% (w/w) of at least one gelling agent selected from the group consisting of carbomers, (f) 2-8% (w/w) of at least one lipid forming the oily phase of the emulsion-gel, (g) 1-5% (w/w) of at least one non-ionic surfactant, and (h) a basic agent selected from the group consisting of ammonia, sodium hydroxide and potassium hydroxide to adjust the pH of the total composition to 6.5-8.

WO2006134406 discloses a gel composition for the topical, local administration of diclofenac through the skin comprises diclofenac sodium in a concentration of about 1% and a mixture of propylene glycol and methocel in a ratio between 6 and 2. The composition also comprises pharmaceutically acceptable excipients.

WO2004/057950 discloses a topical formulation for treating lameness, navicular syndrome, osteoarthritis or a combination thereof in a horse comprising about 0.1% to about 5% diclofenac, about 0.5% to about 20% phospholipids, about 0.1% to about 10% vitamin E, about 1% to about 20% alkylane glycol, and about 1% to about 50% (Cl-C6) alcohol. More in particular, the formulation can comprise about 1% diclofenac salt, about 5% propylene glycol, about 6% ethanol, about 1% vitamin E acetate, about 10% phospholipid, and about 77% water.

WO01/12229 discloses a pharmaceutical formulation for oral or topical administration, comprising an effective amount of one or more hydrophobic active ingredient, such as diclofenac, together with glycerol and polyglicerol derivative, in the form of a dispersion in water of particles having gel-like properties.

WO2008/004231 discloses a pharmaceutical formulation comprising diclofenac sodium added to a vehicle consisting of 60% xylitol, 2% Solutol HS-15 and water.

WO2006/056889 discloses a pharmaceutical formulation comprising diclofenac sodium added to a vehicle consisting of 60% xylitol, 2% Solutol HS-15, 2% Lutrol (Pluronic) F-68 and water.

EP 420798 discloses a pharmaceutical formulations comprising diclofenac sodium in a quantity of 0.1% w/v (Examples 1, 27 and 28) and pharmaceutical formulations comprising Solutol HS-15 in a quantity of 0.5 or 2% w/v (Examples 5, 10, 13, 14, 15, 16, 19, and 23). EP420798 does not comprise any example containing both diclofenac and Solutol HS-15, irrespective from the quantity. Further, EP420798 does not comprise any example containing more than 0.1% w/v of diclofenac or more than 2% w/v of Solutol HS-15.

A gel topical formulation comprising diclofenac sodium salt is sold in Italy under the tradename Dolaut™. The formulation comprises soybean lecithin as solubilizer and alcohols and glycols as co-solvents. Further details on the formulation can be found in U.S. Pat. No. 5,958,379, which discloses a sprayable liquid pharmaceutical composition containing at least one active substance, at least one gel-forming agent consisting of a phospholipid or a phospholipid mixture, an alcohol or an alcohol mixture easily vaporizable, and water.

### SUMMARY OF THE INVENTION

The Applicant has perceived that in spite of the several efforts made in the art, there is still the need to develop a pharmaceutical formulation for the topical administration of diclofenac having improved permeation and bioavailability properties.

The Applicant has also perceived that there is still the need of a pharmaceutical formulation for the topical administration of diclofenac comprising a high concentration of diclofenac, such as, for example, higher than 2% w/v, and even as high as 4% w/v or more, in the form of a liquid formulation able to be sprayed and/or nebulized on the skin and/or mucous surface to be treated.

The Applicant has found that the above mentioned problems may be overcome by a pharmaceutical formulation comprising an aqueous solution of a pharmaceutically acceptable salt of diclofenac, at least one polyoxyalkylene ester of a hydroxy fatty acid, and water.

The pharmaceutical formulation of the present invention comprises water as the main component.

The Applicant has surprisingly found that the pharmaceutical formulation of the present invention has improved permeation and bioavailability properties.

Moreover, the Applicant has surprisingly found that the pharmaceutical formulation of the present invention is stable, and can be stored for the whole useful life of the product without any separation or precipitation of free diclofenac from the solution.

Further, the Applicant has surprisingly found that the pharmaceutical formulation of the present invention allows to maintain in the solution in a stable manner an amount of diclofenac as high as 4% w/v, and even more.

According to a preferred embodiment, the pharmaceutical formulation of the present invention comprises a co-solvent, preferably selected from the group comprising pharmaceutically acceptable glycols and polyols.

Accordingly, the present invention relates to a pharmaceutical formulation which comprises an aqueous solution comprising from 1% to 5% (w/v) of a pharmaceutically acceptable salt of diclofenac, from 3% to 30% (w/v) of at least one polyoxyalkylene ester of a hydroxy fatty acid, water as the main component, and, optionally, a co-solvent.

### SHORT DESCRIPTION OF THE FIGURES

FIG. 1 shows the diffusion profiles of the pharmaceutical formulations 1, 2, and 3 described in the Examples. The ordinate values represent the permeated cumulative amount expressed in milligram per square centimeter (mg/cm²), the abscissa values represent the elapsed time expressed in hours (h).

### DETAILED DESCRIPTION OF THE INVENTION

The pharmaceutical formulation of the present invention may show one or more of the preferred characteristics hereinafter described.

Advantageously, the pharmaceutically acceptable salt of diclofenac comprises any soluble salt of diclofenac with a pharmaceutically acceptable organic or inorganic base.

Typical examples of pharmaceutically acceptable inorganic bases are hydroxides, carbonates and hydrogen carbonates of ammonium, calcium, magnesium, sodium and potassium, for instance sodium hydroxide, ammonium hydroxide, calcium hydroxide, magnesium carbonate, sodium hydrogen carbonate and potassium hydrogen carbonate.

Typical examples of pharmaceutically acceptable organic bases are arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, N-methylglucamine, glucamine, glucosamine, histidine, N-(2-hydroxyethyl)piperidine, N-(2-hydroxyethyl)pyrrolidine, isopropylamine, lysine, methylglucamine, morpholine, piperazine, piperidine, theobromine, triethylamine, trimethylamine, tripropylamine and tromethamine.

Advantageously, the pharmaceutical formulation of the present invention comprises a diclofenac salt selected from sodium, potassium, pyrrolidine, piperidine, N-hydroxyethylpyrrolidine, N-hydroxyethylpiperidine, triethanolamine, diethanolamine, ethylenediamine and diethylamine salts of diclofenac.

The concentration of pharmaceutically acceptable salt of diclofenac in the pharmaceutical formulation of the present invention is preferably between 1% and 5% (w/v), more preferably between 2% and 4% (w/v). Advantageously, the concentration of diclofenac salt in the pharmaceutical formulation of the present invention is about 4% (w/v).

The expression “% (w/v)” used in the present description means parts by weight (expressed in grams) per 100 parts by volume (expressed in milliliter). Accordingly, an aqueous solution containing, for example, 5% w/v of diclofenac means that 100 ml of aqueous solution contain 5 grams of diclofenac.

Preferably, said at least one polyoxyalkylene ester of a hydroxy fatty acid is obtained from the esterification of a hydroxy fatty acid having from 8 to 30 carbon atoms, preferably from 14 to 24 carbon atoms, with a polyoxyalkylene having a molecular weight ranging from 200 to 6,000, preferably from 400 to 1,500.

Advantageously, said hydroxy fatty acid are selected from the group comprising hydroxycaprylic acid, hydroxycapric acid, hydroxylauric acid, hydroxymyristic acid, hydroxypalmitic acid, hydroxystearic acid, hydroxyarachidic acid, hydroxybehenic acid, hydroxylignoceric acid, hydroxymyristoleic acid, hydroxypalmitoleic acid, hydroxyoleic acid, hydroxylinoleic acid, hydroxylinolenic acid, hydroxyarachidonic acid, hydroxyeicosapentaenoic acid, hydroxyerucic acid, and hydroxydocosahexaenoic acid. Particularly useful hydroxy fatty acid is hydroxystearic acid.

Advantageously, said polyoxyalkylene is selected from the group comprising polyethylene glycol 200 (PEG 200), polyethylene glycol 300 (PEG 300), polyethylene glycol 400 (PEG 400), polyethylene glycol 600 (PEG 600), polyethylene glycol 660 (PEG 660), polyethylene glycol 1000 (PEG 1000), polyethylene glycol 1500 (PEG 1500), polyethylene glycol 3000 (PEG 3000), polyethylene glycol 3350 (PEG 3350), polyethylene glycol 4000 (PEG 4000), polyethylene glycol 6000 (PEG 6000), and mixtures thereof.

According to a preferred embodiment, said polyoxyalkylene comprises polyethylene glycol 400 (PEG 400), polyethylene glycol 600 (PEG 600), polyethylene glycol 660 (PEG 660), polyethylene glycol 1000 (PEG 1000), polyethylene glycol 1500 (PEG 1500), and mixtures thereof.

According to a preferred embodiment of the present invention said at least one polyoxyalkylene ester of a hydroxy fatty acid is selected from the group of Solutol™ HS15 (polyethylene glycol 660 hydroxy stearate), a polyglycol ester of polyethylene glycol and 12-hydroxystearic acid, and mixtures thereof.

Solutol HS-15 is a polyethylene glycol 660 hydroxystearate manufactured by BASF (Parsippany, N.J.). Apart from free polyethylene glycol and its monoesters, diesters are also detectable. According to the manufacturer, a typical lot of Solutol HS-15 contains approximately 30% free polyethylene glycol and 70% polyethylene glycol esters.

The concentration of said at least one polyoxyalkylene ester of a hydroxy fatty acid in the pharmaceutical formulation of the present invention is preferably from 3% to 30% (w/v), more preferably from 5% to 25% (w/v), and most preferably from 10% to 20% (w/v). Advantageously, the concentration of the polyoxyalkylene hydroxy fatty acid ester is about 15% (w/v).

Preferably, said co-solvent is selected from the group comprising pharmaceutically acceptable alcohols and polyols, such as for example, ethanol, 1-propanol, 2-propanol, glycerol, propylen glycol, 1,3-butylene glycol, and mixtures thereof.

Advantageously, the pharmaceutical formulation of the present invention comprises 2-propanol, glycerol, or mixtures thereof. More preferably, the co-solvent used in the pharmaceutical formulation of the present invention is a mixture of 2-propanol and glycerol.

The concentration of said co-solvent in the pharmaceutical formulation of the present invention is preferably from 3% to 30% (w/v), more preferably from 5% to 25% (w/v), and most preferably from 10% to 20% (w/v). Advantageously, the concentration of the co-solvent ranges from 15% to 20% (w/v).

The pharmaceutical formulation of the present invention comprises water as the main component, i.e., an amount of water, expressed as weight percentage, higher than the single amount of each component taken alone, preferably equal to or higher than the total amount of all other components. According to a preferred embodiment, the pharmaceutical formulation of the present invention comprises an amount of water higher than 30% (w/v), more preferably higher than 50% (w/v), and most preferably from 65% to 96% (w/v).

The pH of the pharmaceutical formulation of the present invention is preferably ranging from 7 to 9, more preferably from 7.5 to 8.5.

The pharmaceutical formulation of the present invention may further comprise several additives generally known and used in the art. Such non-essential additives of the pharmaceutical formulation according to the invention are, for example, stabilizers, antioxidants, pH correctors, buffers, surfactants, colorants and/or perfumes.

The pharmaceutical formulation according to the present invention can be formulated into a preparation form which is commonly employed as a preparation form for topical application. Advantageously useful preparation forms include, but are not limited specifically to, various solutions, ointments, creams, sprays, foams, cataplasm plasters, and the like. Topical preparations in the form of solution and spray are particularly preferred.

The pharmaceutical formulation of the present invention can be used as analgesic for the treatment of various types of pain, including both chronic and acute painful episodes, such as, for examples, for the treatment of musculoskeletal and joint disorders, like rheumatoid arthritis, osteoarthritis, and ankylosing spondylitis; periarticular disorders, like bursitis and tendonitis; soft tissue disorders, like sprains and strains; and other painful conditions like those following some surgical procedures.

The following examples will illustrate at least one way of carrying out the invention, without however in any way restricting the matter for which protection is sought which is defined by the annexed claims.

### Example 1

The pharmaceutical formulation 1 was a commercial pharmaceutical formulations sold by Gienne Pharma S.p.A. under the trade name Dolaut™.

The pharmaceutical formulations 2 to 5 contained the ingredients of the following Table 1. All the amounts are expressed in w/v percentage, except water as indicated.

TABLE 1

| | | Formulation | | | |
|---|---|---|---|---|---|
| | Function | 2 | 3 | 4 | 5 |
| Diclofenac | Active | 4.00 | 4.00 | 4.00 | 4.00 |
| sodium salt | ingredient | | | | |
| 2-propanol | Co-solvent | 13 | 13 | 13 | 13 |
| Glycerol | | 5 | 5 | 5 | 5 |
| Solutol HS 15 | Solubiliser | 13 | 15 | 15 | 15 |
| Levomenthol | Fragrance | 0.5 | 0.5 | 0.5 | 0.5 |
| Sodium citrate | Buffer | — | — | — | 2.46 |
| tribasic | | | | | |
| dihydrate | | | | | |
| Citric acid 5% | pH | — | — | — | to desired |
| (w/v) in water | Corrector | | | | pH value |
| solution | | | | | |
| 0.1M | Solvent | — | — | q.s. 100 | — |
| Phosphate | | | | ml | |
| Buffer | | | | | |
| Purified water | | q.s. 100 | q.s. 100 | — | q.s. 100 |
| | | ml | ml | | ml |
| Final pH | | 8.03 | 8.03 | 7.9 | 7.65 |

The pharmaceutical formulations 2 to 5 were prepared by introducing into a vessel all the ingredients, except 2-propanol and glycerol. Under continuous stirring, the resulting mixture was heated up to about 45° C. and maintained at that temperature for about 30 minutes. After that, the resulting mixture was cooled under stirring to 25° C. giving an almost clear solution A. Then, the solution B obtained by mixing 2-propanol and glycerol was slowly added under stirring to solution A. After addition, stirring was continued for about 10 minutes at 25° C., obtaining a clear colorless solution. The pH of formulation 5 was controlled and adjusted to the desired value with a solution of citric acid 5% w/v. The volume was brought to 100 ml with purified water, except formulation 4, wherein a 0.1M phosphate buffer was used.

For comparison of the skin permeation of diclofenac sodium standard diffusion experiments with Franz type diffusion cells through porcine skin were performed.

HPLC Analysis

For the quantification of diclofenac sodium the method described in H. Kahlig, et al, “Rheology and NMR self-diffusion experiments as well as skin permeation of diclofenac-sodium and cyproterone acetate of new gel preparations”, J. Pharm. Sci. 94 288-296 (2005) was used.

Analysis was done by HPLC (Perkin Elmer, US) consisting of an automatic autosampler ISS-200, a pump and an UV-diode array detector. The column (240 mm×4 mm) packed with Nucelosil 100-5C18 was eluted at 45° C. with a mobile phase consisting of acetonitrile and phosphate buffer, pH 5 (40:60, v/v) at a flow rate of 1.0 ml/min. The concentration of diclofenac was established by comparing the peak area of the unknown with a standard calibration curve. Standard solutions contained between 0.23 and 0.014 mg/ml diclofenac. Linear regression analysis of the peak areas gave a correlation coefficient of 0.999. Samples (20 μl) were withdrawn from the receptor chamber and injected directly by the autosampler.

Parameters

Stock solution: 2.281 mg/ml methanol

Column: Nucleosil C18

Mobile phase: acetonitrile/phosphate-buffer pH=5 (40:60/v:v)

UV-detection λmax: 230 nm

Flow rate: 1.0 ml/min

Retention time about 10 min

Skin Preparation

Porcine abdominal skin was shaved and then prepared with a dermatome (GB 228R, Aesculap) set at 1.2 mm. The skin was stored in a freezer at −20° C. until use. Two hours prior to the experiment the samples were thawed.

Diffusion Cell Preparation

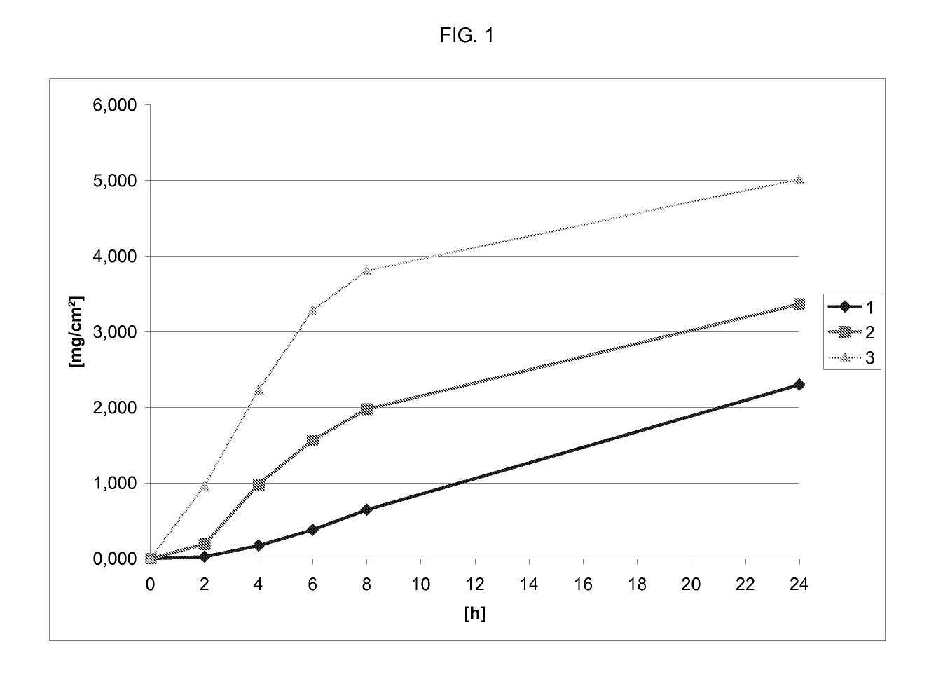

Standard diffusion experiments using Franz-type diffusion cells (Permegear, USA) with about 1 cm² of permeation area and porcine skin were performed. The receptor compartment was filled with 2 ml of 0.012 M phosphate buffer (pH 7.4). Excised skin was mounted in the cell, stratum corneum uppermost, with the dermal side facing the receptor compartment. The diffusion cells were thermostated at skin surface temperature of 32° C. and stirred by magnetic bars. At defined time intervals (2, 4, 6, 8 and 24 h) the acceptor medium was removed for analysis and replaced with fresh acceptor medium. If necessary, a suitable dilution with acceptor medium was additionally performed. Each amount of applied formulation was equivalent to 10 mg diclofenac sodium. Three parallel tests were performed for each product. The following Tables and FIG. 1 illustrate the average results and standard deviation of the tests.

TABLE 2
Formulation 1

| | Average | Standard | Average | Standard |
|---|---|---|---|---|
| Hours | (mg/cm²) | Deviation | (%) | Deviation |
| 0 | n.a. | n.a. | n.a. | n.a. |
| 2 | 0.026 | 0.023 | 0.259 | 0.225 |
| 4 | 0.174 | 0.116 | 1.690 | 0.136 |
| 6 | 0.382 | 0.207 | 3.714 | 2.049 |
| 8 | 0.646 | 0.292 | 6.266 | 2.925 |
| 24 | 2.300 | 0.469 | 22.221 | 5.092 |

TABLE 3
Formulation 2

| | Average | Standard | Average | Standard |
|---|---|---|---|---|
| Hours | (mg/cm²) | Deviation | (%) | Deviation |
| 0 | n.a. | n.a. | n.a. | n.a. |
| 2 | 0.192 | 0.053 | 1.820 | 0.546 |
| 4 | 0.982 | 0.654 | 9.394 | 6.354 |
| 6 | 1.567 | 0.853 | 14.977 | 8.326 |
| 8 | 1.977 | 0.922 | 18.878 | 9.051 |
| 24 | 3.366 | 0.289 | 32.009 | 3.397 |

TABLE 4
Formulation 3

| | Average | Standard | Average | Standard |
|---|---|---|---|---|
| Hours | (mg/cm²) | Deviation | (%) | Deviation |
| 0 | n.a. | n.a. | n.a. | n.a. |
| 2 | 0.963 | 1.299 | 8.929 | 12.012 |
| 4 | 2.231 | 0.346 | 20.756 | 3.975 |
| 6 | 3.287 | 0.491 | 30.497 | 5.121 |
| 8 | 3.811 | 0.530 | 35.322 | 5.180 |
| 24 | 5.017 | 0.286 | 46.428 | 1.042 |

The results of formulations 4 and 5 substantially confirmed the results of formulation 3. The data of tables 2 to 4 clearly shown that the formulations 2 and 3 of the present invention have demonstrated an improved permeation. The amount of permeated diclofenac of formulation 2 is almost one fold and half the amount of formulation 1, and the amount of formulation 3 is more than two folds.

A sample of formulation 5 was packaged in 25 ml amber glass bottles. The sample was submitted to an accelerated aging test of six month at 40° C. and 75% of relative humidity and to a long term aging test of one year at 25° C. and 60% of relative humidity. No degradation or separation of solid particles were observed at the end of the test. Moreover, the assay (HPLC) of the active pharmaceutical ingredient (API) complied with the ICH (www.ich.org) stability testing specifications (±5% of the nominal amount).

1. A pharmaceutical formulation which comprises an aqueous solution comprising from 1% to 5% (w/v) of a pharmaceutically acceptable salt of diclofenac, from 3% to 30% (w/v) of at least one polyoxyalkylene ester of a hydroxy fatty acid, and water as the main component.

2. The pharmaceutical formulation according to claim 1, wherein said formulation further comprises a co-solvent.

3. The pharmaceutical formulation according to claim 1, wherein said at least one polyoxyalkylene ester of a hydroxy fatty acid is obtained from a process comprising esterifying a hydroxy fatty acid having from 8 to 30 carbon atoms with a polyoxyalkylene having a molecular weight ranging from 200 to 6,000.

4. The pharmaceutical formulation according to claim 3, wherein said hydroxy fatty acid has from 14 to 24 carbon atoms, and said polyoxyalkylene has a molecular weight ranging from 400 to 1,500.

5. The pharmaceutical formulation according to claim 1, wherein said hydroxy fatty acid is at least one selected from the group consisting of hydroxycaprylic acid, hydroxycapric acid, hydroxyl auric acid, hydroxymyristic acid, hydroxypalmitic acid, hydroxystearic acid, hydroxyarachidic acid, hydroxybehenic acid, hydroxylignoceric acid, hydroxymyristoleic acid, hydroxypalmitoleic acid, hydroxyoleic acid, hydroxylinoleic acid, hydroxylinolenic acid, hydroxyarachidonic acid, hydroxyeicosapentaenoic acid, hydroxyerucic acid, and hydroxydocosahexaenoic acid.

6. The pharmaceutical formulation according to claim 3, wherein said polyoxyalkylene is at least one selected from the group consisting of polyethylene glycol 200 (PEG 200), polyethylene glycol 300 (PEG 300), polyethylene glycol 400 (PEG 400), polyethylene glycol 600 (PEG 600), polyethylene glycol 660 (PEG 660), polyethylene glycol 1000 (PEG 1000), polyethylene glycol 1500 (PEG 1500), polyethylene glycol 3000 (PEG 3000), polyethylene glycol 3350 (PEG 3350), polyethylene glycol 4000 (PEG 4000), polyethylene glycol 6000 (PEG 6000), and mixtures thereof.

7. The pharmaceutical formulation according to claim 3, wherein said at least one polyoxyalkylene ester of a hydroxy fatty acid is selected from the group consisting of Solutol™ HS15 (polyethylene glycol 660 hydroxy stearate), polyglycol ester of polyethylene glycol and 12-hydroxystearic acid, and mixtures thereof.

8. The pharmaceutical formulation according to claim 1, wherein said salt of diclofenac is selected from the group consisting of sodium, potassium, pyrrolidine, piperidine, N-hydroxyethylpyrrolidine, N-hydroxyethylpiperidine, triethanolamine, diethanolamine, ethylenediamine and diethylamine salts of diclofenac.

9. The pharmaceutical formulation according to claim 2, wherein said co-solvent is selected from the group consisting of pharmaceutically acceptable alcohols and polyols.

10. The pharmaceutical formulation according to claim 9, wherein said co-solvent is selected from the group consisting of ethanol, 1-propanol, 2-propanol, glycerol, propylen glycol, 1,3-butylene glycol, and mixtures thereof.

11. The pharmaceutical formulation according to claim 10, wherein said co-solvent is selected from the group consisting of 2-propanol, glycerol, or mixtures thereof.

12. The pharmaceutical formulation according to claim 1, wherein said formulation comprises an amount of said pharmaceutically acceptable salt of diclofenac ranging from 2% to 4% (w/v).

13. The pharmaceutical formulation according to claim 1, wherein said formulation comprises an amount of said at least one polyoxyalkylene ester of a hydroxy fatty acid ranging from 5% to 25% (w/v).

14. The pharmaceutical formulation according to claim 1, wherein said formulation comprises an amount of said co-solvent ranging from 3% to 30% (w/v).

15. The pharmaceutical formulation according to claim 14, wherein said formulation comprises an amount of said co-solvent ranging from 10% to 20% (w/v).

16. The pharmaceutical formulation according to claim 1, wherein said formulation has a pH value ranging from 7 to 9.

17. The pharmaceutical formulation according to claim 1, wherein said formulation is selected from the group of topical formulations including solutions, ointments, creams, sprays, foams, and cataplasm plasters.
